(19) United States
(12) Patent Application Publication
(10) Pub. No.: US 2020/0247911 A1
(43) Pub. Date: Aug. 6, 2020

(54) Purification of Bacterial Polysaccharides

(71) Applicant: MSD Wellcome Trust Hilleman Laboratories PVT. LTD., New Delhi (IN)

(72) Inventors: Sandeep Sharma, Uttar Pradesh (IN); Nitin KUMAR, Uttar Pradesh (IN); Sarmad HANIF, New Delhi (IN); Manoj Kumar CHHIKARA, Delhi (IN); Davinder GILL, New Delhi (IN)

(21) Appl. No.: 16/628,413

(22) PCT Filed: Apr. 27, 2018
(86) PCT No.: PCT/IN2018/050260

§ 371 Date: Jan. 3, 2020

(30) Foreign Application Priority Data
May 17, 2017 (IN) 201711017277
Apr. 5, 2018 (IN) 201812013041

(51) Int. Cl. C08B 37/00; A61K 39/095

(52) U.S. Cl. CPC C08B 37/0003; A61K 39/095

(57) ABSTRACT

The present invention relates to rapid purification of Neisseria meningitidis serogroup W and serogroup Y polysaccharides. The N. meningitidis polysaccharides of the present invention are capable of being used in the production of economical polysaccharide protein conjugate vaccine(s) against meningococcal infections.

### RELATED APPLICATIONS

This application U.S. National stage entry of International Application No. PCT/IN2018/050260, which designated the United States and was filed on Apr. 27, 2018, published in English which claims priority to Indian Application No. 201711017277, filed May 17, 2017 and Indian Application No. 201812013041, filed Apr. 5, 2018. The entire teachings of the above applications are incorporated herein by reference.

### FIELD OF THE INVENTION

The present invention relates to an improved process of purification of bacterial polysaccharide. The present invention particularly relates purification of Neisseria meningitidis serogroup W and serogroup Y polysaccharides. The N. meningitidis polysaccharides of the present invention are capable of being used in the production of economical polysaccharide protein conjugate vaccine(s) against meningococcal infections.

### BACKGROUND OF THE INVENTION

Neisseria meningitidis, often referred to as meningococcus, is a Gram-negative bacterium that can cause meningitis and other forms of meningococcal disease such as meningococcemia.

On the basis of the type of capsular polysaccharide present on N. meningitidis (Men), thirteen serogroups have been identified and among the 13 identified capsular types of N. meningitidis, six (A, B, C, W135, X, and Y) account for most meningococcal disease cases worldwide. MenA has been the most prevalent in Africa and Asia, but is rare/practically absent in North America. In the Europe and United States, serogroup B (MenB) is the predominant cause of disease and mortality, followed by serogroup MenC and MenW. In recent past, MenX outbreaks have started showing up in sub-Saharan Africa. The multiple serogroups have hindered development of a universal vaccine for meningococcal disease.

The production of the first meningitis polysaccharide vaccine was accomplished in 1978 as there was an urgent need to combat this fatal disease. Later it was observed that the plain polysaccharide based vaccines were not very efficient in children below two years of age. These observations led to further research which revealed that infants have an immature immune system and cannot elicit immune response against plain polysaccharides.

The immune response may be characterized as T-cell dependent (TD) immune response and T-cell independent (TI) immune response. Proteins and peptides are known to elicit TD antigens by stimulating the helper T lymphocytes and generating memory cells. In contrast, polysaccharides belong to the TI antigens which do not induce T-cell activation and do not form any memory B cells, which is a major drawback while dealing with infants as they have an immature immune system.

Thus, there was a need for conjugating the bacterial polysaccharide to a protein carrier which induces a T-cell-dependent immune response characterized by increased immunogenicity among infants, prolonged duration of protection and in the reduction of nasopharyngeal carriage of meningococci. This need was fulfilled by ingenious research resulting in the production of polysaccharide-protein conjugate vaccines and the first meningococcal conjugate vaccine was licensed in United Kingdom in 1999.

The polysaccharides, especially antigenic polysaccharides, used in preparation of vaccines may be monovalent, bivalent and poly (multi) valent vaccines containing one, two or more polysaccharides, respectively. These are readily available in the market for prevention of certain diseases or infections caused by various microorganisms. The multivalent polysaccharide vaccines have been used for many years and have proved valuable in preventing diseases such as Pneumococcal, Meningococcal or Haemophilus influenzae diseases.

The production of purified N. meningitidis capsular polysaccharides is the foremost requirement for an effective conjugation with the carrier protein and its development as a conjugate vaccine. The cost for the cultivation of N. meningitidis and the purification of polysaccharides is generally high and involves long working hours since it involves a series of production and purification steps.

Improvement in the production and purification steps would lead to formulation of efficacious and economically viable conjugate vaccines.

There are a number of patents and non-patent disclosures that describe the processes of production and purification of polysaccharides. One such disclosure is the US2009/0182128 A1, Paolo Costantino et al, relates to purification of Men W, A, and Y using CTAB and ethanol treatment (50%-95%) and use of CaCl2 and carbon filtration. The disclosed patent application uses multiple steps for the purification of crude polysaccharides and long hours for the purification process.

Another patent application no. U.S. Ser. No. 12/041,745 discloses a method of producing a meningococcal meningitis vaccine, the method, includes culturing N. meningitidis to produce capsular polysaccharides of serogroups A, C, Y and W-135 in N. meningitidis fastidious medium (NMFM), isolating the capsular polysaccharides from the culture, purifying the capsular polysaccharides of any residual cellular biomass; and depolymerizing the capsular polysaccharide mechanically. The cited art utilizes approximately 43 hours of purification process. The purification is achieved by mechanical means such as sonication. Also, the yield of the purified capsular polysaccharide as achieved in cited prior art is 43 mg/L for Men Y and 47 mg/L for MenW which is comparatively much lesser than what is achieved in the present invention.

Presently, the various methods used for the polysaccharide purification of N. meningitidis serogroups take relatively long purification time and complex process steps thereby increasing the cost of production and making the process commercially less feasible since they cannot be scaled up in a cost-effective and timely manner.

It is an object of the present invention to provide improved purification process of N. meningitidis serogroup W and Y polysaccharides at reduced time and with high yield. Said improvements will result in manufacturing polysaccharide protein conjugate vaccine at lesser price and subsequently vaccine can be made available to children of developing countries at an affordable rate.

### OBJECT OF THE INVENTION

The main object of the present invention is to provide a process of purification of bacterial capsular polysaccharides.

Another object of the present invention is to provide a process of purification of Neisseria meningitidis serogroup W and serogroup Y polysaccharides.

Yet another object of the present invention is to purify Neisseria meningitidis serogroup W and serogroup Y polysaccharides, while eliminating impurities in a very short time by simple, efficient, improved and commercially scalable methods.

Yet another object of the present invention is to produce high quality product with better yield that meet the relevant quality specifications.

### SUMMARY OF THE INVENTION

The present invention describes a rapid, industrially scalable, cost effective process for production of bacterial polysaccharide preferably Neisseria meningitidis. The said process provides a purification method for purifying N. meningitidis polysaccharide (PS) at a significantly reduced time. Most part of the purification process can be completed at room temperature and the whole process does not require any chromatography step.

The present invention describes purification steps for producing high yields of N. meningitidis serogroup W and Y capsular polysaccharides. The crude polysaccharide from the fermentation broth is subjected to concentration and diafiltration against MilliQ water (MQW) to form a concentrate with reduced impurity level. The concentrate so obtained is subjected to treatment with an alkali such as NaOH at 1±0.2 M at predetermined temperature for an optimized time. The resultant partially purified polysaccharide is subjected again to diafiltration with MQW followed by carbon filtration and finally subjected to sterile filtration.

The purified polysaccharide is thus recovered in a significantly reduced time using a scalable, cost-effective and efficient method.

The process of instant invention can be used to purify meningococcal polysaccharides. The process exhibits a number of advantages over prior art, such as providing a robust and rapid method of producing purified polysaccharides meeting the desired specifications with better yields. An additional advantage is that this process is entirely scalable.

### BRIEF DESCRIPTION OF DRAWINGS

FIG. 1 depicts the process flow for the MenW and MenY polysaccharide Purification.

FIGS. 2 (a) and (b) depicts the HPLC Chromatogram of MenW and MenY polysaccharide, respectively.

FIGS. 3 (a) and (b) depicts the NMR spectrum of MenW and MenY polysaccharide, respectively.

FIG. 4 depicts the percentage inhibition of anti-MenW polyclonal antibodies with Standard MenW polysaccharide and purified MenW polysaccharide batches.

### DETAILED DESCRIPTION OF THE INVENTION

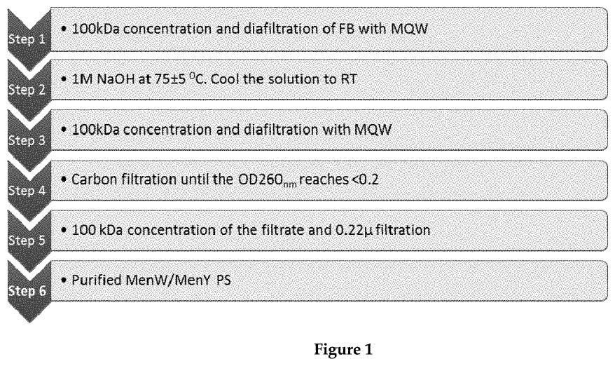

The invention discloses steps that have been optimized to enable the purification of MenW and MenY polysaccharides in lesser time as shown in FIG. 1.

The fermented broth of MenW and MenY polysaccharide is concentrated and diafiltered with 100 KDa (0.1 m²) polyethersulfone (PES) membrane. The resultant MenW and MenY concentrate so obtained is treated with alkali. More preferably, the MenW and MenY concentrate is treated with 1±0.2 M NaOH for time period in the range of 2±0.5 hours, preferably 2 hours at a temperature range of 75±5° C.

The partially purified polysaccharide so obtained is cooled to room temperature. After cooling, the partially purified polysaccharide is concentrated and diafiltrated with 100 kDa PES membrane with 20±2 volumes of MQW. This step of concentration and diafitration is done to eliminate the NaOH. The step of concentration and diafitration is followed by carbon filtration with Millistak carbon filters (MILISTAK® Pod Depth Filters, 0.027 m² or higher scale) until the partially purified polysaccharide reaches to an optical density of ≤0.2.

The filtrate so obtained is again concentrated with 100 KDa PES membrane and is subjected to sterile filtration with 0.2 μm PES assembly to get the purified MenW and MenY polysaccharides.

The purified polysaccharide so obtained is stored at −20±2° C. for further use. The purified polysaccharide qualified various desired specifications with a yield of up-to 174 mg/L and 405 mg/L of fermentation harvest for MenW and MenY, respectively.

Examples 1 to 3 of the invention details the method of carrying out the invention. The method employed in example 1 and 2 result in PS which does not meet the specifications in the intermediate steps and PS loss was observed over the different steps and low PS yields were detected in the end. Therefore, the protocols were not considered as option for PS purification, while the example 3 discloses the best mode of carrying out the invention, wherein purified PSs so obtained qualified desired specifications with a yield of up to 174 mg/ml and 405 mg/L for MenW PS and MenY PS, respectively. Also, the purification is completed rapidly in a short duration of 7±1 hours.

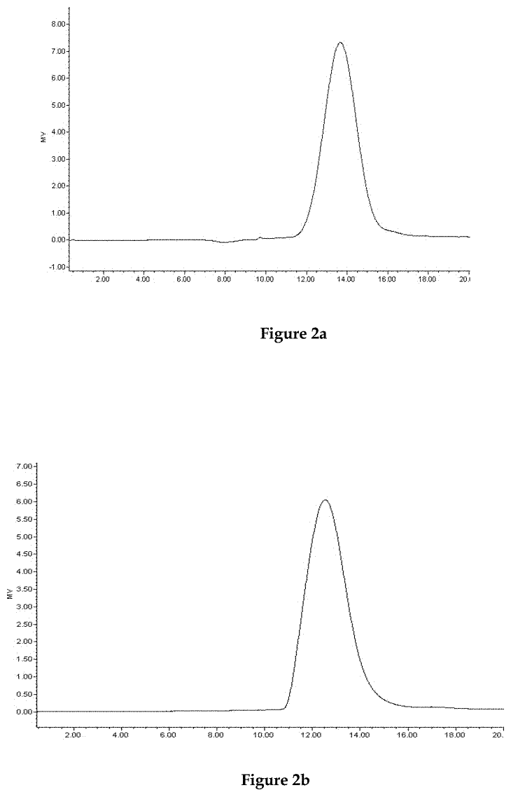

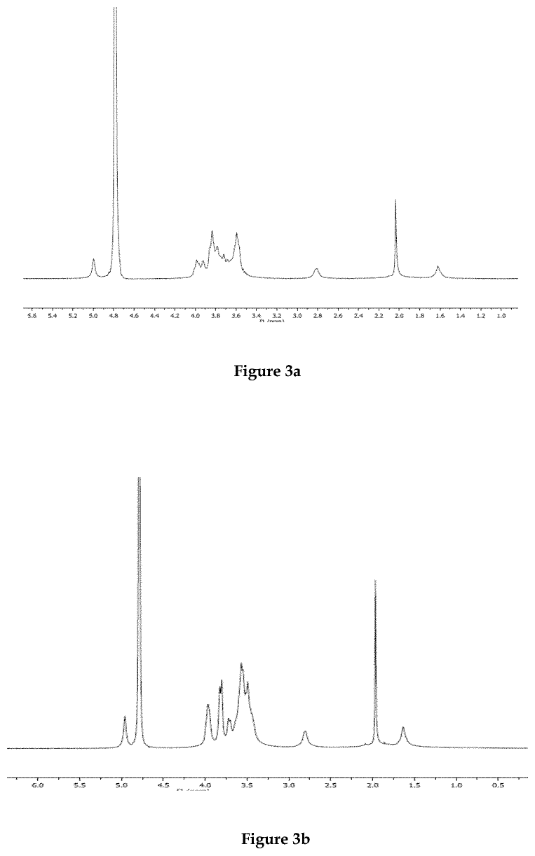

The analysis of the purified MenW and MenY is done by HPLC and ¹H-NMR spectrum for purity and identity analysis and the results of the same is shown in FIGS. 2 and 3, respectively.

The corroboration of the above employed procedures can be conveniently understood from Table 1 and 2 which clearly show that the purified polysaccharides met the desired standard specifications.

### Purified MenW and MenY Polysaccharides Specifications are Shown Below in Table-1 and Table-2

TABLE 1
MenW polysaccharide control tests

| Parameters | Specification | Batch 1 | Batch 2 | Batch 3 | Batch 4 | Batch 5 | Average | SD |
|---|---|---|---|---|---|---|---|---|
| Identity | Positive reaction | Positive | Positive | Positive | Positive | Positive | — | — |
| | with specific | | | | | | | |
| | antibody | | | | | | | |
| Sialic acid (%) | ≥56 | 73.4 | 73.6 | 65.8 | 63.2 | 65.4 | 68.3 | 4.9 |
| Mol. Weight (kD) | To report the | 308 | 292 | 347 | 308 | 308 | 312.6 | 20.4 |
| | result | | | | | | | |
| PS content (mg/ml) | To report the | 1.39 | 1.03 | 1.1 | 0.6 | 0.45 | 0.9 | 0.4 |
| | result | | | | | | | |
| Nucleic Acid | ≤2 | 1.63 | 1.71 | 0.9 | 0.62 | 1.25 | 1.2 | 0.5 |
| content (%) | | | | | | | | |
| Protein content (%) | ≤5 | 3.14 | 4.72 | 3.6 | 0.9 | 0.5 | 2.6 | 1.8 |
| Endotoxin (EU/μg) | <100 | 0.72 | 0.96 | 0.09 | 0.3 | 0.02 | 0.4 | 0.4 |
| O-acetyl | De-O-acetylated | De-O-Ac | De-O-Ac | De-O-Ac | De-O-Ac | De-O-Ac | De-O-Ac | |
| Molecular size | ≥80% of PS | 86.66 | 87.23 | 89.38 | 90.61 | 91.09 | 89.0 | 2.0 |
| distribution (%) | should elute | | | | | | | |
| | before K_{D} 0.50 | | | | | | | |
| PS yield/L (mg) | To report the | 166.8 | 144.2 | 173.8 | 92.4 | 63.9 | 128.2 | 48.1 |
| | result | | | | | | | |

EU: Endotoxin Unit;
SD: Standard deviation

TABLE 2
MenY polysaccharide control tests

| Parameters | Specification | Batch 1 | Batch 2 | Batch 3 | Batch 4 | Average | SD |
|---|---|---|---|---|---|---|---|
| Identity | Positive reaction | Positive | Positive | Positive | Positive | — | — |
| | with specific | | | | | | |
| | antibody | | | | | | |
| Sialic acid (%) | ≥56 | 58 | 63 | 67 | 59 | 61.8 | 4.1 |
| Mol. Weight (kD) | To report the | 806 | 814 | 630 | 621 | 717.8 | 106.6 |
| | result | | | | | | |
| PS content (mg/ml) | To report the | 2.65 | 4.13 | 2.2 | 1.4 | 2.6 | 1.1 |
| | result | | | | | | |
| Nucleic Acid | ≤2 | 1.76 | 2.54 | 1.32 | 1.4 | 1.8 | 0.6 |
| content (%) | | | | | | | |
| Protein content (%) | ≤5 | 2.6 | 1.18 | 0.94 | 1.12 | 1.5 | 0.8 |
| Endotoxin (EU/μg) | <100 | 3 | 8 | 2.6 | 4.9 | 4.6 | 2.5 |
| O-acetyl | De-O-acetylated | De-O-Ac | De-O-Ac | De-O-Ac | De-O-Ac | De-O-Ac | — |
| Molecular size | ≥80% of PS | 93 | 96 | 99 | 99 | 96.8 | 2.9 |
| distribution (%) | should elute | | | | | | |
| | before K_{D} 0.50 | | | | | | |
| PS yield (mg/L) | To report the | 344.5 | 404.7 | 343.2 | 218.4 | 327.7 | 78.3 |
| | result | | | | | | |

EU: Endotoxin Unit;
SD: Standard deviation

Various Aspects of the Invention Described in Detail Above is Now Illustrated with Non-Limiting Examples as Discussed Below:

### Example-1

### MenW Polysaccharide (PS) Purification Using Phenyl Sepharose.

The fermented broth (FB) is 100 kDa concentrated and diafiltered with 10-12 volumes of MilliQ water (MQW). After the above process of diafiltration and concentration, the polysaccharide is treated with 2M NaOH for 2±0.5 hours at 75±5° C. The PS is then cooled to room temperature. After cooling, the 100 kDa concentration and diafiltration of the crude polysaccharide is performed with 20 volumes of 20 mM Tris HCl (pH 8±0.2). Thereafter, 20% w/v ammonium sulfate is added to the concentrated and diafiltered PS. It is then loaded on phenyl sepharose resin using XK16/20 column. Flow through is collected, followed by a wash of 5-10 column volume (CV) with equilibration buffer (20 mM Tris HCl (pH 8±0.2) and 20% ammonium sulfate). The collected material is then 30 kDa concentrated and diafiltered with 20 volumes of MQW followed by 0.2μ filtration.

### Example-2

### MenW PS Purification Using Sodium Hydroxide (NaOH), Ethanol, CTAB, Sodium Deoxycholate, Sodium Acetate.

The FB is 100 kDa concentrated and diafiltered with 10-12 volumes of MilliQ water (MQW). After the diafilteration and concentration, the partially purified PS is treated with 1M NaOH for 2±0.5 hours at 75±5° C. The PS so obtained is cooled to room temperature. After cooling, the concentration and diafiltration of the PS is performed through 100 KDa PES membrane (0.1 m²) with 20 volumes of MQW, followed by ethanol precipitation using 100% v/v absolute ethanol with overnight incubation at 2-8° C. Centrifugation at 10550×g is done next day and the collected pellet is dissolved in MQW. To this 80% v/v of absolute ethanol is added with continuous mixing for 2±0.5 hours at RT (25±2° C.). Centrifugation at 10550×g is done and collected pellet is dissolved in MQW. This is followed by treatment with 12% v/v of 10% Hexadecyltrimethylammonium bromide (CTAB) stock solution with an overnight stirring/mixing at room temperature (RT). Centrifugation at 10550×g is done and the pellet so collected is dissolved in MQW and 40% v/v absolute ethanol, with the addition of 1% w/v sodium deoxycholate (DOC) and 8% w/v sodium acetate to the dissolved pellet. The resultant solution is kept with continuous stirring for 2 hours at RT. Centrifugation at 10550×g is done and supernatant so obtained is collected and further diafiltered and concentrated with 100 kDa followed by 0.2μ filtration.

### Example-3

### MenW or MenY PS Purification Using NaOH Treatment and Carbon Filtration:

Fermentation broth of MenW or MenY is concentrated and diafiltered with 100 kDa (0.1 m²) PES membrane. 2.5 L of fermentation working volume is used in the process of purification for Neisseria meningitidis serogroup W and serogroup Y The concentrate of MenW/MenY so obtained is subjected to alkali treatment with 1±0.2 M NaOH for 2±0.5 hours at 75±5° C. The partially purified polysaccharide so obtained are then cooled to room temperature. After cooling, the concentration and diafiltration of the partially purified PS is performed through 100 KDa PES membrane with 20±2 volumes of MQW to eliminate out NaOH followed by carbon filtration with MQW primed Millistak carbon filters (Millistak+® Pod Depth Filter, 0.027 m²) until the OD₂₆₀ₙₘ≤0.2 is achieved. The collected filtrate is concentrated with 100 kDa PES membrane and is sterile filtered with 0.2 μm PES assembly to get the purified MenW or MenY PS. The purified polysaccharide is stored at −20±2° C. for further use. Purified PSs yield of up to 174 mg/ml and 405 mg/L for MenW and MenY, respectively. Further, the PSs so obtained qualified for desired specifications as disclosed in Table 1 and Table 2.

FIGS. 2 (a) and (b) show HPLC-SEC chromatogram of representative purified MenW and MenY polysaccharide using RI detector, respectively.

The HPLC-SEC analysis of the purified polysaccharides is done using TSK 4000-5000 PWXL HPLC columns in series and monitored by RI detector using sodium nitrate as running buffer. The single major peak in HPLC-SEC chromatogram and other physico-chemical analyses (Table 1, 2) confirm the purity of the MenW and MenY polysaccharides to the desired levels.

FIGS. 3 (a) and (b) shows 1H-NMR spectrum of representative purified MenW and MenY polysaccharide, respectively. The 1H-NMR for purified MenW and MenY polysaccharide is recorded on Bruker Avance 500 MHz instrument using deuterium oxide (D2O) as a solvent. The spectral peaks in FIG. 3 (a) confirm the identity of the MenW polysaccharide. The peak at 2 ppm in the spectrum corresponds to the three protons of CH3 group from N-acetyl group (NH—Ac) present in the polysaccharide monomer structure. The peak at 1.6 ppm represents the axial H-3 proton and peak at 2.8 ppm represents the equatorial H-3 of the sialic acid ring. The broad multiplet at 3.5-4.1 ppm corresponds to the all other protons on the sialic acid and galactose ring. The peak at 5 ppm corresponds to the alpha H-1 of the galactose ring.

The spectral peaks in FIG. 3(b) confirm the identity of the MenY polysaccharide. The peak at 1.9 ppm in the spectrum corresponds to the three protons of CH3 group from N-acetyl group (NH—Ac) present in the polysaccharide monomer structure. The peak at 1.6 ppm represents the axial H-3 proton and peak at 2.8 ppm represents the equatorial H-3 proton of the sialic acid ring. The broad multiplet at 3.3-4.1 ppm corresponds to the all other protons on the sialic acid and glucose ring. The peak at 5 ppm corresponds to the alpha H-1 of the glucose ring. The spectrum shows no major peak of impurity indicating the purity of the polysaccharide.

### Example-4: MenW and MenY PS Identity by Inhibition ELISA

The purified sample containing meningococcal capsular polysaccharide of serogroup W or Y is incubated with the specific polyclonal antibody (primary antibody) so that complexes is formed between the antibody and antigens in the sample. These complexes are then added to a container in which competitor antigens are immobilized. Antibody which is not complexed with epitopes from the polysaccharide sample bind to these immobilized competitor antigens. The antibody which is bound to the immobilized competitor antigens (after usual washing steps, etc.) is then detected by adding an enzyme labelled secondary antibody which binds to the primary antibody. The label is used to identify the reaction of immobilized primary antibody to secondary antibody utilizing a chromogenic substrate. The reduction in the absorbance in test well as compared to the control well (without any test sample) confirms the presence of the specific antigen in the test sample.

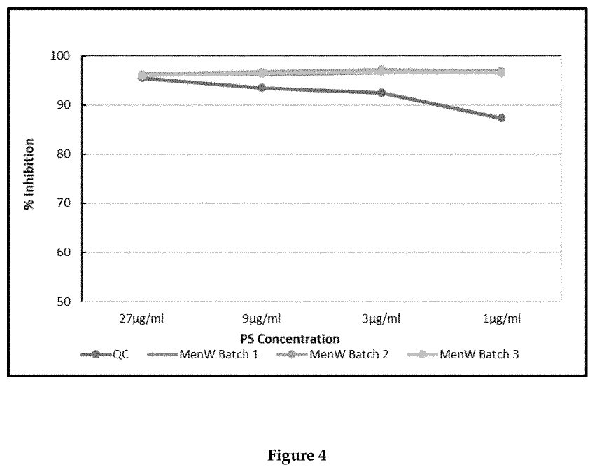

Briefly, the ELISA Plate A is coated with 100 μl of coating solution having equal volume of in-house PS and mHSA and incubated for overnight at 2-8° C. A no-antigen-control was included as control. The coated plate is blocked at room temperature with 200 μl of blocking buffer. Quality control polysaccharide (NIBSC Standard) of defined concentration are serially diluted three-fold as are the bacterial culture supernatant (test samples) and incubated in Plate B with serogroup specific commercially available polyclonal primary antibody for 1 hour at 37° C. The antigen-antibody mixture from Plate B is transferred to blocked Plate A and further incubated for two hours (1.5 hours at 37° C. and half an hour at room temperature). The reaction is further incubated with optimized secondary antibody dilution for 1 hour and reaction is developed using 100 μl of TMB substrate and incubated for 10 min. The reaction is stopped with 50 μl of 2M H2SO4 per well before optical density (OD) at 450 nm is observed with reference to 630 nm. The inhibition percentage is calculated from inhibition of OD in standard or test sample dilutions in relation to OD of no-antigen control wells. The representative PS concentration versus percent inhibition curves for MenW identity assay is presented in FIG. 4 which show that the MenW polysaccharides purified using the current invention inhibit serogroup specific antibodies as good as the polysaccharide standard and hence confirm the identity of the polysaccharide.

We claim:

1. A process for purifying N. meningitidis serogroup W (MenW) and serogroup Y (MenY) capsular polysaccharides, wherein the said process comprising the steps of:
(a) concentration and diafiltration of fermentation broth;
(b) alkali treatment of concentrated and diafiltered concentrated broth of step (a) at high temperature to obtain partially purified polysaccharide;
(c) concentration and diafiltration of partially purified polysaccharide of step (b) and carbon filtration to obtain filtrate;
(d) sterile filtration of filtrate as obtained in step (c) to obtain purified MenW or MenY polysaccharide;
wherein said process of purification is completed rapidly within 7±1 hours.

2. The process as claimed in claim 1, wherein said concentration and diafiltration of step (a) is carried with 100 KDa PES membrane.

3. The process as claimed in claim 1, wherein said alkali treatment of step (b) is carried with 1±0.2 M NaOH for 2±0.5 hours at 75±5° C.

4. The process as claimed in claim 1, wherein said concentration and diafiltration of partially purified polysaccharide of step (c) is carried with 100 KDa PES membrane followed by carbon filtration using Millistak Pod depth filters.

5. The process as claimed in claim 1, wherein said sterile filtration of step (d) is carried with 0.2 μm PES membrane.

6. The process as claimed in claim 1 wherein said process yields purified polysaccharides meeting desired quality standards.

7. The process as claimed in claim 1, wherein yield of purified polysaccharide of Neisseria meningitidis serogroup W and Y are up to 174 mg/ml and 405 mg/L fermentation broth, respectively.

8. The process as claimed in claim 1, wherein the complete process of purification is carried within 6 to 8 hours.

9. The process of the purification as claimed in claim 1, wherein said process results in N. meningitidis serogroup W (MenW) and serogroup Y (MenY) capsular polysaccharides which are capable of being used in the production of economical polysaccharide protein conjugate vaccine(s) against meningococcal infections.
